(19) United States
(12) Patent Application Publication
(10) Pub. No.: US 2022/0062392 A1
(43) Pub. Date: Mar. 3, 2022

(54) COLLECTING PHYSICAL THERAPY INFORMATION TO ENHANCE TREATMENT EFFICACY OF BOTULINUM TOXIN

(71) Applicant: Ipsen Biopharm Limited, Wrexham (GB)

(72) Inventor: Jean-Michel Gracies, Wrexham (GB)

(73) Assignee: Ipsen Biopharm Limited, Wrexham (GB)

(21) Appl. No.: 17/447,545

(22) Filed: Sep. 13, 2021

Related U.S. Application Data
(63) Continuation of application No. 16/088,488, filed on Sep. 26, 2018, now abandoned.
(60) Provisional application No. 62/313,418, filed on Mar. 25, 2016.

(51) Int. Cl. A61K 38/48; G16H 20/17; A61K 9/00; A61K 35/74

(52) U.S. Cl. CPC A61K 38/4893; G16H 20/17; G16H 20/30; A61K 35/74; A61K 9/0019

(57) ABSTRACT

Methods for improving active movement capacity in a subject experiencing muscle impaired or abnormal activity, and software products and computer systems for implementing such methods. A subject having impaired active movement capacity is administered an effective amount of botulinum toxin and instructed to undergo physical therapy, such as through a Guided Self-rehabilitation Contract (GSC), and to record information associated with the physical therapy into, e.g., a software program running on a computer device. The information may be stored in a database, which can be accessed by a medical practitioner who is administering the botulinum toxin. This information is used to improve the clinical management of the subject's impaired active movement capacity.

### FIELD OF THE INVENTION

The present invention relates generally to improving active movement capacity in a subject, and more particularly, to using a prescribed physical therapy regimen in conjunction with a Clostridial neurotoxin, such as botulinum toxin, to reduce symptoms associated with impaired or abnormal active movement capacity.

### BACKGROUND

Abnormal movement capacity includes muscle rigidity or stiffness, tightness, contracture, spasms (i.e., jerky involuntary movements), dystonia, hypertonia, and clonus (i.e., repetitive involuntary movement or reflex). Abnormal movement capacity is commonly associated with neurological disorders, including stroke, cerebral palsy, muscular dystrophy, spinal cord injury, brain injury, spastic disorders, such as blepharospasm, spasmodic torticollis (cervical dystonia), oromandibular dystonia and spasmodic dysphonia (laryngeal dystonia), and neurodegenerative diseases, such as multiple sclerosis and Parkinson's disease. It can also be associated with various other metabolic disorders, muscle diseases, upper or lower motor neuron lesions, and Guillain-Barré syndrome.

In addition to potentially causing pain, the symptoms associated with abnormal muscle tone can interfere with a person's voluntary movement and ability to carry out daily activities. Physical therapy with stretching and active exercise is a recognized treatment for abnormal muscle tone. Additional information regarding how physical therapy can be useful for treating movement capacity caused by neurologic movement disorders is described in Veerbeck, Janne Marieke et al., “What Is the Evidence for Physical Therapy Poststroke? A Systematic Review and Meta-Analysis,” PLOS One, vol. 9:2 (2014), the disclosure of which is incorporated herein by reference.

In addition to physical therapy, application of a toxin, such as botulinum toxin, to the affected muscles can be helpful. For example, when injected into a muscle, the botulinum toxin binds rapidly and strongly to presynaptic cholinergic nerve terminals and inhibits the exocytosis of acetylcholine from the nerve ending to the muscle fiber. This results in partial paralysis, and hence relaxation, of the muscle afflicted by spasm.

Botulinum toxin can be used to treat various types of neurologic conditions, including movement disorders associated with injury or disease of the central nervous system (such as trauma, stroke, multiple sclerosis, Parkinson's disease, and cerebral palsy), cervical dystonia (spasmodic torticollis), blepharospasm, hyperhidrosis, chronic daily headache, strabismus, esophageal achalasia, and focal dystonia. For additional information regarding how botulinum toxin can be useful in the treatment of spasticity, reference is made to Simpson D. M. et al, “Assessment: Botulinum neurotoxin for the treatment of spasticity (an evidence-based review): Report of the Therapeutics and Technology Assessment Subcommittee of the American Academy of Neurology,” Neurology 6; 70(19), 1691-1698 (2008), the disclosure of which is incorporated herein by reference.

### SUMMARY OF THE INVENTION

A method is provided for improving active movement capacity in a subject. The method comprises: administering to a subject in need thereof a first botulinum toxin treatment to a muscle affected by impaired active movement capacity. In some embodiments, the method further comprises instructing the subject to undergo physical therapy comprising a physical activity involving the affected muscle. In an embodiment, the method further comprises instructing the subject to record information associated with the physical therapy. In some embodiments, the physical therapy information is recorded by the subject, for example into a log or diary. In other embodiments, the physical therapy information is entered into a software program that operates on a computer device and the physical therapy information is stored in a database. In some aspects, the subject enters the physical therapy information into the log or diary, or into the software program. The botulinum toxin treatment(s) may be administered or received into the affected muscle, for example by injection. The treatment(s) may also be administered or received into a neuromuscular junction of the affected muscle, for example by injection.

The method may further comprise: accessing the subject's physical therapy information; designing a treatment regimen based, at least in part, on the subject's physical therapy information; and administering a second botulinum toxin treatment to the subject in accordance with the treatment regimen, wherein the botulinum toxin treatment is sufficient to treat the subject's impaired active movement capacity.

In some embodiments, the physical therapy information is received by a software program that operates on a first computer device and physical therapy information is stored in a database on the first computer device or a second computer device that is in communication with first computer device. In some embodiments, the subject enters the physical therapy information into the software program.

In some embodiments, the method may further comprise: reviewing the subject's physical therapy information (e.g. by accessing the physical therapy information stored in a database); designing a treatment regimen based, at least in part, on the subject's physical therapy information; and administering a second botulinum toxin treatment to the subject in accordance with the treatment regimen, wherein the botulinum toxin treatment is sufficient to treat the impaired active movement capacity.

In another embodiment, in a method for improving active movement capacity in a subject using botulinum toxin, the improvement consists of: determining the appropriate botulinum toxin treatment based on a medical practitioner's assessment of how the subject has responded to a prescribed physical therapy regimen, the physical therapy regimen comprising: (i) administering or receiving botulinum toxin to improve active movement capacity; (ii) instructing the subject to engage in, or engaging in, a physical activity involving a muscle affected by impaired active movement capacity; and (iii) instructing the subject to record, or recording, information associated with the physical therapy. In some embodiments, the physical therapy information is received by a software program that operates on a computer device and the physical therapy information is stored in a database associated with the computer device. In some embodiments, the subject enters the physical therapy information into the software program.

In another embodiment, the method comprises receiving a first botulinum toxin treatment to improve active movement capacity in a muscle affected by impaired active movement capacity; undergoing physical therapy comprising a physical activity involving the affected muscle; and recording information associated with the physical therapy. In some embodiments, the physical therapy information is received by a software program that operates on a first computer device and the information is stored into a database on the first computer device or a second computer device that is in communication with first computer device. In some embodiments, the subject enters the physical therapy information into the software program. The method may further comprise receiving a second botulinum toxin treatment, wherein the second treatment is determined, at least in part, on the physical therapy information.

In another embodiment of the invention, a computer system is provided. The computer system is programmed to perform steps of a computer-implemented method, the method comprising: receiving from a subject undergoing physical therapy and botulinum toxin treatment for impaired active movement capacity, information associated with the physical therapy; storing the physical therapy information into a database; and transmitting the physical therapy information to a medical practitioner. In one embodiment, the medical practitioner has previously administered the botulinum toxin treatment to the subject.

In another embodiment, a non-transitory computer-readable storage medium storing instructions is provided. The non-transitory computer-readable medium, when executed by a computer system, causes the computer system to perform steps of a computer-implemented method, the method comprising: receiving from a subject undergoing physical therapy and botulinum toxin treatment for impaired active movement capacity, information associated with the physical therapy; storing the physical therapy information into a database; and transmitting the physical therapy information to a medical practitioner. In one embodiment, the medical practitioner has previously administered the botulinum toxin treatment to the subject.

### DESCRIPTION OF THE DRAWINGS

FIG. 1 shows a subject 10 being administered a dose of botulinum toxin by injection 12.

FIG. 2 shows the subject 10 entering physical therapy information into a tablet computer 14, which is in communication with a host server computer 22 via a network link 20.

FIG. 3 shows a medical practitioner 30 operating a computer 32 that is in communication via a communication link 24 that is in communication with a host computer system 22 that stores the information entered by the subject.

### DETAILED DESCRIPTION OF THE INVENTION

The present invention is useful in the clinical management of a subject having impaired active movement capacity. Impairments in active movement capacity may be the result, for example, of abnormal muscle overactivity in the subject. The subject may have any of the various types of medical conditions associated with impaired active movement capacity, including stroke, cerebral palsy, muscular dystrophy, spinal cord injury, brain injury, spastic disorders, such as blepharospasm, spasmodic torticollis (cervical dystonia), oromandibular dystonia and spasmodic dysphonia (laryngeal dystonia), and neurodegenerative diseases, such as multiple sclerosis and Parkinson's disease.

The affected muscle can be anywhere in the subject's body, including in the upper limbs (adult or pediatric) such as the shoulders, arms, or hands; in the lower limbs (adult or pediatric), such as in the leg or foot; or in the bladder (e.g., as affected in neurogenic detrusor overactivity (NDO). The term “affected muscle” as used herein refers to any muscle affected by impaired active movement capacity. With the proper treatment, the subject may experience various improvements in active movement capacity, such as increased mobility, increased flexibility, increased strength, increased passive or active range of motion in the affected limbs, reduced pain, and ability to independently perform activities of daily living.

In the present invention, a subject is administered botulinum toxin in conjunction with specifically prescribed physical therapy techniques. The administration of botulinum toxin is typically performed by a medical professional. As used herein, the term “medical professional” includes a clinician, physician, nurse, medical technician, or the like. In some embodiments, the subject may self-administer the botulinum toxin. The botulinum toxin can be administered in any suitable manner, such as by transdermal administration or injection into the affected muscle(s) or the neuromuscular junction of the affected muscle(s).

In the present invention, the botulinum toxin-producing strain is preferably Clostridium botulinum, but is not limited thereto, and it will be apparent to those skilled in the art that any strain capable of producing a botulinum toxin may be used in the present invention. As used herein, the term “botulinum toxin” is meant to include not only a neurotoxin produced by the Clostridium botulinum strain, but also any modified, recombinant, hybrid, fusion, and chimeric botulinum toxins. A modified or recombinant botulinum toxin may have a light chain and/or heavy chain produced by non-Clostridium species in a recombinant manner. In addition, the term “botulinum toxin” as used herein is meant to include any and all known botulinum toxin serotypes, including serotypes A, A1, A2, A3, A4, B, C, C1, D, E, F and G, as well as botulinum toxin complexes (e.g., 300, 600 and 900 kDa complexes), and a pure botulinum toxin (e.g., a 150 kDa neurotoxic molecule), which are all useful in the practice of the present invention. For additional information regarding the properties of the various botulinum toxins, reference is made to Simpson D. M. et al, “Assessment: Botulinum neurotoxin for the treatment of spasticity (an evidence-based review): Report of the Therapeutics and Technology Assessment Subcommittee of the American Academy of Neurology,” Neurology 6; 70(19), 1691-1698 (2008), the disclosure of which is incorporated herein by reference.

In one embodiment of the invention, the botulinum toxin administered to the subject is type A toxin. Botulinum toxin type A1 complex is marketed, e.g., under the trade names DYSPORT®, XEOMIN®, CORETOX®, RELOXIN®, and BOTOX®. In another embodiment of the invention, the botulinum toxin used is type B toxin, e.g., as marketed under the trade names MYOBLOC® and NEUROBLOC®. In other embodiments of the invention, the botulinum toxin used is any of the other known toxin types, including A1, A2, A3, A4, C, C1, D, E, F or G.

Botulinum toxin is obtained commercially by establishing and growing cultures of C. botulinum in a fermenter, and harvesting and purifying the fermented mixture in accordance with known techniques. The “A” form of botulinum toxin is currently available commercially from several sources, for example, from Ipsen Biopharmaceuticals Limited under the tradename DYSPORT®, from Merz Pharma under the tradename XEOMIN®, from Medytox Inc. under the tradename CORETOX®, and from Allergan Inc. under the tradename BOTOX®.

The biological activity of botulinum toxin relates, e.g., to inhibition of neurotransmission over the synapse at the neuromuscular junction, leading to muscle paralysis or inhibition of exocytosis, in particular exocytosis of acetylcholine or of another neurotransmitter. The biological activity of botulinum neurotoxin is linked to its proteolytic activity. One way to determine the biological activity of any botulinum toxin is, therefore, to measure the proteolytic activity on the relevant substrate mentioned above. Assays that can be used to determine this activity are known in the art; one such assay is described in WO 95/33850, the disclosure of which is hereby incorporated by reference.

The botulinum toxin may be administered by any means known in the art, including injection directly into an affected muscle (i.e., intramuscular injection), injection into the neuromuscular junction of the affected muscle, sub-cutaneous injection, or transdermal administration.

Targeting of neuromuscular junctions can increase the effectiveness of the botulinum toxin treatment and/or allow for use of lower concentrations dosages of botulinum toxin, as described in Gracies J M, Lugassy M, Weisz D J, Vecchio M, Flanagen S, Simpson D M, “Botulinum toxin dilution and endplate targeting in spasticity: a double-blind controlled study,” Arch Phys Med Rehabil 2009, 90: 9-16. the disclosure of which is hereby incorporated by reference. In some embodiments, the botulinum toxin is injected into a neuromuscular junction.

Transdermal administration allows the toxin to be delivered to a target site associated with impaired active movement capacity to provide a therapeutic effect, such as a relaxation of the muscle or a decrease in muscle spasticity, without the difficulty and discomfort associated with needle injection of the botulinum toxin. If desired, adhesive patches containing amounts of a botulinum toxin sufficient to improve active movement capacity can be self-administered by the subject based on a medical practitioner's instructions. Use of an adhesive patch for transdermal delivery of a therapeutic drug is described, for example, in Tonnesen, P. et al., “A double blind trial of a 16-hour transdermal nicotine patch in smoking cessation,” New Eng J Medicine, 325(5); 311-315: August 1991, the disclosure of which is hereby incorporated by reference.

The botulinum toxin can be presented as a sterile pyrogen-free aqueous solution or dispersion, or as a sterile powder for reconstitution into a sterile solution or dispersion. If desired, tonicity adjusting agents, such as sodium chloride, glycerol and/or various sugars can be added. Stabilizers may be included if desired. The formulation may be preserved by means of any suitable pharmaceutically acceptable preservative, such as a paraben.

In some embodiments, the botulinum toxin is formulated in unit dosage form, for example, as a sterile solution in a vial, or as a vial or sachet containing a lyophilized powder for reconstituting in a suitable carrier, such as saline, for injection. In one aspect, the botulinum toxin is formulated in a solution containing saline and pasteurized human serum albumin, which stabilizes the toxin. The solution may be sterile filtered, filled into individual vials, and then vacuum dried to give a sterile lyophilized powder. In use, the powder can be reconstituted by the addition of sterile unpreserved normal saline (sodium chloride 0.9% for injection). In another aspect, the commercially available sterile botulinum toxin powder can be incorporated into the polymeric matrix of a suitable carrier using known methodologies, and formed into an adhesive patch for use in conjunction with a skin permeation enhancer such as dimethyl sulfoxide (DMSO) or Azone (1-dodecylazacycloheptan-2-one).

Typically, the amount of the botulinum toxin administered to the subject is sufficient to improve the impairment in active movement capacity in the one or more affected muscles. The amount of the botulinum toxin will depend upon a variety of factors, including the severity of the condition, the number of muscle groups requiring treatment, the age, size, and/or gender of the subject, and the type and potency of the particular toxin. The potency of the toxin may be expressed as a multiple of the LD₅₀ value. One LD₅₀ unit is the equivalent amount of toxin which causes the death of 50% (one-half) of a group of test animals, such as laboratory mice. Alternative methods of determining the potency of the toxin may also be employed, including, for example, any method included in the European Pharmacopoeia monograph 01/2005:2113, which is hereby incorporated by reference.

The dose administered to the subject is in an amount effective to improve active movement capacity. For example, the amount may be between 0.01 and 1000 units of botulinum toxin, whatever the type of botulinum toxin or whatever its provenance. Smaller or larger doses may be administered in appropriate circumstances. In some embodiments, the dosage amount of the botulinum toxin is from about 1 to about 500 units per muscle injection. For example, the dosage amount could be about 1 unit, 50 units, 100 units, about 150 units, about 200 units, about 250 units, about 300 units, about 350 units, about 400 units, about 450 units, or about 500 units. In one embodiment, a subject with impaired active movement capacity (e.g., a patient having spastic paresis) is administered up to 1000 units of abobotulinum toxin A (DYSPORT®). Additional information regarding appropriate dosage amounts of botulinum toxin are described in the publications by Jabeen, Afshan et al., “Guidelines for the use of botulinum toxin in movement disorders and spasticity,” Ann Indian Acad Neurol., vol. 14 (Suppl 1), pp. S31-S34: July 2011 and by Ozcakir, Suheda et al., “Botulinum Toxin in Poststroke Spasticity,” Clin Med Res., vol. 5(2), pp. 132-138: June 2007, the disclosures of which are incorporated herein by reference.

Depending on the potency of the botulinum toxin, and its duration of action, the doses may need to be administered intermittently. Ultimately, however, both the quantity of toxin administered, and the frequency of its administration will be at the discretion of the medical practitioner(s) responsible for the treatment, and will be commensurate with questions of safety and the effects produced by the toxin.

Recent studies have demonstrated that subjects experiencing post-stroke spasticity can be benefited by receiving early botulinum toxin treatment. For example, see Verplancke et al., “A randomized controlled trial of botulinum toxin on lower limb spasticity following acute acquired severe brain injury,” Clin Rehabil 2005; 19:117-125; Cousins et al., “Does low-dose botulinum toxin help the recovery of arm function when given early after stroke? A phase II randomized controlled pilot study to estimate effect size,” Clin Rehabil 2010; 24:501-513; Hesse et al., “An early botulinum toxin A treatment in subacute stroke patients may prevent a disabling finger flexor stiffness six months later: a randomized controlled trial,” Clin Rehabil 2012; 26:237-245; Rosales et al., “Botulinum toxin injection for hypertonicity of the upper extremity within 12 weeks after stroke: A randomized controlled trial,” Neurorehabil Neural Repair 2012; 26:812-821; and Fietzek et al., “Early botulinum toxin treatment for spastic pes equinovarus—a randomized double-blind placebo-controlled study,” Eur J Neurol 2014; 21:1089-1095; the disclosures of which are each incorporated herein by reference. Accordingly, in some embodiments of the method, a subject experiencing symptoms of impaired active movement capacity (e.g., spasticity) arising from a stroke receives early botulinum toxin treatment, e.g., within one year of the stroke. For example, a subject who has suffered a stroke may be administered his or her first botulinum toxin treatment within 9 months, 6 months, 3 months, 2 months, 1 month, 3 weeks, 2 weeks, 1 week, or even a few days after suffering the stroke.

Successful treatment of impaired active movement capacity with botulinum toxin can be determined using routine methods known to persons of ordinary skill in the art. For example, successful treatment can be associated with improved active movement capabilities, reduced muscle tone, reduced pain, reduced spasticity, reduced deformity, and the like. The determination can be made by the subject, one or more medical professionals, or a combination of the two.

In the present invention, in conjunction with the botulinum toxin treatment, the subject engages in physical therapy. The physical therapy comprises one or more physical activities or techniques involving the affected muscle, such as exercising, stretching, contracting and relaxing, joint mobilization, myofascial release techniques, heating or cooling the affected muscle, training the affected muscle, performing alternating movements of the affected muscle, casting/orthotics, and/or positioning the affected muscle (e.g., prone lying, standing, or sitting). Additional information regarding the types of physical therapy activities that can be useful for treating neurologic movement disorders are described in Veerbeck, Janne Marieke et al., “What Is the Evidence for Physical Therapy Poststroke? A Systematic Review and Meta-Analysis,” PLOS One, vol. 9:2, c87987: February 2014, the disclosure of which is incorporated herein by reference.

In some embodiments, a treatment regimen will be determined for the subject. As used herein, the term “treatment regimen” means the one or more types of treatments the subject is undergoing to improve active movement capacity, including botulinum toxin treatments, physical therapy, or a combination of the two.

In one embodiment, the treatment regimen will be determined based, at least on part, on the personal work and the regular reports by the subject (e.g., a patient). In one aspect, the subject is treated according to a Guided Self-rehabilitation Contract (GSC). This strategy aims to generate and maintain patient motivation, so as to enable long-term and intense use of physical therapy techniques, such as stretching and training. This may produce substantial functional improvements in chronic stages. In such contracts, the therapist acts as a coach, providing for example: technical guidance by selecting and teaching required exercises to the patient in infrequent, thorough visits (for example, every month) for a duration of at least one year; and psychological support to encourage compliance by the patient. In some embodiments, the subject visits the medical practitioner at least two or more times over the course of one year; in some cases, three or more times over the course of one year; in some cases, four or more times over the course of one year; in some cases, five or more times over the course of one year, and in some cases, six or more times over the course of one year.

The patient, in turn, agrees to perform the prescribed daily stretch postures and maximal amplitude alternating movements over the long term and documents this work in a written diary. To facilitate such contracts, a manual and an application for cell-phones and tablets can be used. Additional information regarding the strategy of Guided Self-rehabilitation Contracts is described in Gracies et al., “Contrat d'Autorééducation Guidéé dans la parésie spastique,” Association Neuroloco, Paris, ISBN 978-2-35327-169-6 (2013) and Gracies et al., “The Concept of Guided Self-Rehabilitation Contracts in the Treatment of Deforming Spastic Paresis, Physikalische Medizin Rehahilitationsmedizin Kurortmedizin 25(03) (2015), the disclosures of which are hereby incorporated.

In some embodiments, the subject is under regular guidance by a medical practitioner and/or therapist. The medical practitioner and/or therapist may select and teach the physical activity to the subject. The medical practitioner and/or therapist may also offer encouragement to the subject, which in turn leads to improved adherence to the physical therapy and contributes to the subject's improved active movement capacity.

In some embodiments, the method of the invention comprises administering to the subject a first botulinum toxin treatment to a muscle affected by impaired active movement capacity; and instructing the subject to undergo physical therapy that comprises a physical activity involving the affected muscle. Another embodiment of the present invention is an improvement to this method which comprises: instructing the subject to record information associated with the physical therapy. In some embodiments, the physical therapy information is recorded (e.g., by the subject) into a log or diary. In other embodiments, the physical therapy information is recorded (e.g., by the subject) into a software program. The software program may operate on a first computer device and the physical therapy information is stored in a database on the first computer device or a second computer device that is in communication with first computer device.

In some embodiments, the method further comprises: reviewing the subject's physical therapy information (e.g., by accessing the physical therapy information stored in a database); designing a treatment regimen based, at least in part, on the subject's physical therapy information; and administering a second botulinum toxin treatment to the subject in accordance with the treatment regimen, wherein the botulinum toxin treatment is sufficient to treat the impaired active movement capacity.

In another embodiment, in a method for improving active movement capacity in a subject using botulinum toxin, the improvement consists of: determining the appropriate botulinum toxin treatment based on an assessment of how the subject has responded to a prescribed physical therapy regimen, the physical therapy regimen comprising: (i) administration of botulinum toxin to improve active movement capacity in a muscle affected by impaired active movement capacity; (ii) engagement in at least one physical activity involving the affected muscle; and (iii) recordation of information associated with the physical therapy. In some embodiments, the physical therapy information is recorded into a log or diary. In other embodiments, the physical therapy information is recorded into or by a software program that operates on a computer device and the physical therapy information is stored in a database associated with the computer device. In some embodiments, the subject records the physical therapy information.

In another embodiment, the method comprises: receiving a first botulinum toxin treatment to improve active movement capacity in a muscle affected by impaired active movement capacity; undergoing physical therapy that comprises at least one physical activity involving the affected muscle; and recording information associated with the physical therapy. In some embodiments, the physical therapy information is recorded by the subject into a log or diary. In other embodiments, the physical therapy information is recorded into or by a software program that operates on a first computer device and the information is stored into a database on the first computer device or a second computer device that is in communication with first computer device. The method may additionally comprise receiving a second botulinum toxin treatment, wherein the second treatment is determined, at least in part, on the physical therapy information.

In some embodiments of the invention, the clinical management of the subject's impaired active movement capacity is enhanced by virtue of the fact that information about the physical therapy is being recorded into a log or diary, or into a software program that is able to receive and communicate information about the physical therapy. In some aspects of the invention, the subject's motivation is improved as a result of recording the physical therapy information. As used herein, the term “motivation” refers to the subject's desire or willingness to continue to engage in physical therapy. The act of recording the physical therapy information may, for example, result in the subject being more likely to continue to engage in physical therapy, or to engage in more physical therapy, or for a longer period of time. In some embodiments, the act of recording the physical therapy information results in the subject actually engaging in more physical therapy than he or she would have engaged in had he or she not recorded the physical therapy information. In some embodiments, the subject's improved motivation resulting from recording the physical therapy information is responsible, at least in part, for the improvement in the subject's active movement capacity.

In some aspects, the improvement in the subject's active movement capacity is substantial. Techniques for measuring improvement in active movement capacity are well known in the art, and include, for example, the Modified Ashworth Scale (MAS), Tardieu Scale (TS), and Triple Spasticity Scale (TSS). Additional information regarding how to measure active movement capacity is described in Li et al, “Reliability of a new scale for measurement of spasticity in stroke patients,” J. Rehabil. Med. 46(8), 746-53 (2014), the disclosure of which is herby incorporated by reference.

In some embodiments, the physical therapy is tailored to the particular subject. In some embodiments, the subject is instructed to undergo physical therapy and/or is instructed to record information associated with the physical therapy at a regular interval, such as three times a day, twice a day, once a day (daily), twice a week, or weekly. As a result, the subject may engage in physical therapy and/or record the information associated with the physical therapy at a regular interval, such as three times a day, twice a day, once a day (daily), twice a week, or weekly.

In other embodiments of the invention, a software program is used in conjunction with the botulinum toxin treatment and/or physical therapy, such as the GSC strategy. In these embodiments, a medical practitioner and/or therapist instructs the subject to use a software program that receives information associated with the physical therapy. The software program is running on any suitable computer device. Various sorts of information may be received by the software program, such as the types of physical therapy activities performed, duration of the physical therapy activities, frequency of the physical therapy activities, symptoms relating to the impaired active movement capacity (e.g., amount of pain or stiffness, frequency of muscle spasms, duration of muscle spasms, range of motion, etc.), the effect (if any) of the physical therapy activities, and any other comments about the physical therapy activities. The software program receives the information that is associated with the subject's physical therapy and stores that information into a database. The database can reside on the same computer device used by the subject or on a different computer device that is able to communicate with the computer device used by the subject.

In some embodiments, the medical practitioner reviews the physical therapy information. Using a computer device that is able to communicate with the database, the medical practitioner can access the physical therapy information. In one aspect, after reviewing the information, the medical practitioner (e.g., clinician) uses this information to determine an appropriate treatment regimen, or to make or suggest an adjustment to an existing treatment regimen. In another aspect, the medical practitioner uses the physical therapy information to determine or vary the botulinum toxin treatment, i.e., the mode, amount, or frequency of botulinum toxin administered to the subject. This determination will take into account various factors, including the physical therapy information, to arrive at an optimal treatment regimen for that subject.

As used herein, the term “optimal treatment regimen” means the treatment regimen determined by the medical practitioner to be optimal for a particular subject based on a variety of factors, including the age, size, and/or gender of the subject, the muscle group(s) requiring treatment, the potency of the toxin, the physical therapy activities performed by the subject, the duration and frequency of those physical therapy activities, the subject's symptoms relating to the impaired active movement capacity, and the information associated with the physical therapy activities. The optimal treatment regimen can comprise administration of botulinum toxin to the affected muscle, prescription of physical therapy activities (e.g., within a GSC strategy), or a combination of the two. In one aspect, the optimal treatment regimen comprises a prescription for physical therapy followed by administration of botulinum toxin in an amount sufficient to improve active movement capacity in the one or more affected muscles.

As used herein, the term “computer device” refers to any electronic device for storing and processing data, typically in binary form, according to instructions given to it in a software program, and includes, for example, a desktop, laptop, or tablet personal computers; “netbooks”; mobile communication devices, such as smartphones; personal digital assistants; portable audio or video file players; portable game players; portable electronic readers; or equivalent devices. The computer device can be in communication with another computer device by any suitable type of network (such as internet), and can use any suitable protocol, medium (e.g. fiber optic, coaxial cable, wireless broadband, etc.), network interface, or bandwidth.

The computer device used by the subject can be the same or different from the computer device used by the medical practitioner to access the physical therapy information. In some embodiments, the computer device used by the subject is the same computer device that the medical practitioner uses to access the physical therapy information. For example, the subject may enter the physical therapy information into his/her smartphone and bring that smartphone to the clinic visit to have the information viewed by the medical practitioner directly from the smartphone. In other embodiments, the computer device that the medical practitioner uses to access the physical therapy information is different from the computer device used by the subject to enter the physical therapy information. For example, the subject may enter the physical therapy information into a website portal that is specially designed to collect this type of information and the medical practitioner uses his/her own desktop computer to access the website portal and view the information that the subject has entered. In another example, the subject may be wearing a portable electronic device (e.g., a smart-watch or body-mounted exercise tracker), which records the subject's physical therapy activity and transmits the information to the subject's computer device.

The computer device on which the database resides may be the same or different from the computer device used by the subject to receive the physical therapy information and/or the computer device used by the medical practitioner to access that information. In some embodiments, the computer device on which the database resides is different from both the computer device used by the subject to receive the physical therapy information and the computer device used by the medical practitioner to access that information. For example, the subject may enter the information into a website portal via their laptop computer. The information is stored on a web server computer, which is then accessed by the medical practitioner on his/her own computer to view the physical therapy information.

In some embodiments, the invention is a software product. As used herein, the term “software product” refers to a non-transitory computer-readable storage medium storing instructions that when executed by a computer system, causes the computer system to perform the recited steps. The software product may reside on any suitable computer-readable storage medium, such as CD-ROM, DVD, memory, hard disk, flash drive, RAM, ROM, cache, and the like. The software platform for implementing the present invention can vary depending on design considerations such as user preference, cost, implementation, case of use, machine capabilities, network limitations, etc.

In some embodiments, a computer system is provided. The computer system is programmed to perform steps of a computer-implemented method, the system comprising: receiving from a subject undergoing physical therapy and botulinum toxin treatment for impaired active movement capacity, information associated with the physical therapy; storing the physical therapy information into a database; and transmitting the physical therapy information to a medical practitioner who has administered the botulinum toxin treatment to the subject.

In some embodiments, the invention is a computer system comprising one or more computer devices that are programmed to perform the methods of the present invention. The hardware platforms used by the subject, the medical practitioner, and/or any other third parties may be different, but operate together as a system. For example, the subject being treated could use his/her own computer device to enter the physical therapy information, that information could be stored on a different computer device located remotely (e.g., a third party web server), and the medical practitioner could use his/her own computer device to access the information. In this scenario, these three computer devices can be considered to operate together as a system. The physical and/or functional components of the computer system may be distributed, centralized, or arranged in any suitable manner. Communications between the different physical and/or functional component may be performed in any suitable way. Moreover, the present invention encompasses all the various ways in which the operating work may be divided among different physical and/or functional components.

In some embodiments, the physical therapy information is received by the computer system from a first computer device, the database resides on a second computer device, and the physical therapy information is transmitted by the computer system to a third computer device. The computer devices may be separate. The first computer device may be in communication with the second computer device and the second computer device may be in communication with the third computer device. In one aspect, the first computer device is programmed to receive the physical therapy information from the subject, the database resides on a second computer device, and the third computer device receives the transmitted physical therapy information.

In other embodiments, a non-transitory computer-readable storage medium storing instructions is provided. The non-transitory computer-readable medium, when executed by a computer system, causes the computer system to perform steps of a computer-implemented method, the method comprising: receiving from a subject undergoing physical therapy and botulinum toxin treatment for impaired active movement capacity, information associated with the physical therapy; storing the physical therapy information into a database; and transmitting the physical therapy information to a medical practitioner who has administered the botulinum toxin treatment to the subject.

To assist in understanding the present invention, a particular embodiment is described in detail with references to the figures.

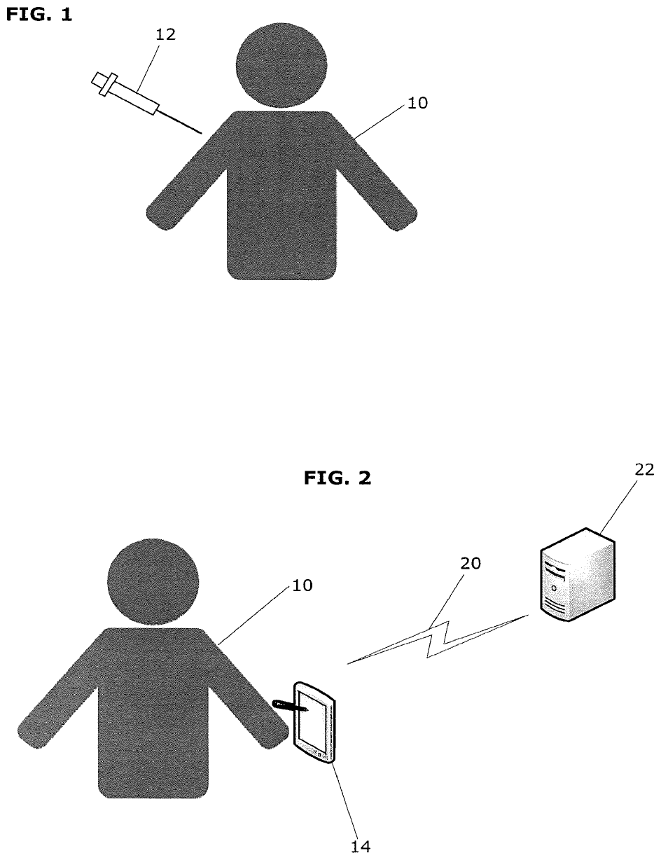

FIG. 1 shows a subject 10 being administered a dose of botulinum toxin by injection (e.g., by a syringe needle 12) into an overactive muscle. This injection could be performed in any suitable clinical setting, such as at a hospital or in an outpatient clinic. The medical practitioner 30 determines which muscle(s) need to be injected, the dosage amount, etc. If needed, multiple injections can be performed.

In conjunction with the botulinum toxin treatment (e.g., after administering the injection), the medical practitioner 30 instructs the subject 10 to undergo physical therapy activities and to use a computer software program to enter information associated with those physical therapy activities. The subject 10 undergoes the physical therapy as instructed by the medical practitioner 30. The physical therapy can be performed in any suitable manner, such as being performed by a physical therapist, or being performed under the supervision or guidance of a physical therapist, or performed directly by the subject 10 himself/herself (e.g., the subject 10 self-performs exercises, stretching, etc. at home), or a combination thereof.

FIG. 2 shows the subject 10 entering information associated with the physical therapy into a tablet computer 14. Installed on the tablet computer 14 is an application program that provides a user interface for the subject 10 and that receives the information entered by the subject 10. The tablet computer 14 is in communication with a host server computer 22 (e.g., operated by the supplier of the software program, or the supplier of the botulinum toxin, or another third party) via a network link 20 (e.g., connection through WiFi and then through a home internet connection). The information entered by the subject 10 is transmitted to the host server computer 22 and stored in a database residing therein.

In one embodiment, the subject's 10 interaction with the software program alone is sufficient to enhance the efficacy of the treatment. For example, in the GSC strategy, the act of entering information associated with the physical therapy activities performed by the subject 10 could give the subject 10 a feeling of accomplishment and encourage him/her to continue with the physical therapy activities, thereby enhancing the efficacy of the treatment. In an alternate embodiment, the physical therapy information is reviewed by the medical practitioner 30, who may then use that information to determine a course of treatment, or make or suggest an adjustment to an existing course of treatment.

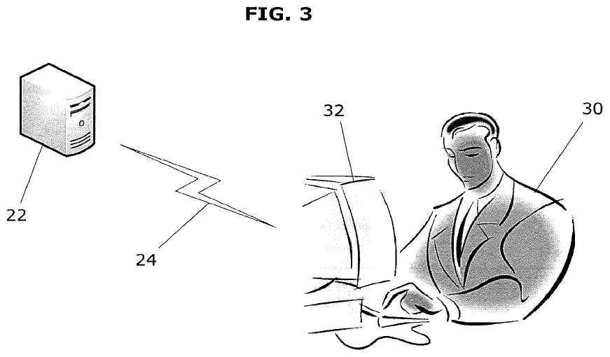

FIG. 3 shows a medical practitioner 30 working on a personal computer 32 that is in communication via a communication link 24 (e.g., by an office internet connection) with the host server computer 22. Using the personal computer 32, the medical practitioner 30 is able to access the subject's 10 physical therapy information stored on host server computer 22 and view the information. In an alternate embodiment, the subject 10 could bring the tablet computer 14 to the medical practitioner 30, who then views the information directly on the tablet computer 14.

Based on review of this information, the medical practitioner 30 may perform any suitable action to improve the clinical management of the subject's 10 impaired active movement capacity. For example, the medical practitioner could inform the subject 10 that he/she is pleased to see the progress made by the subject 10 and give encouragement to continue with the physical therapy. In an alternate embodiment, the medical practitioner could use this information to make decision(s) about further treatment of the subject with botulinum toxin. For example, the subject 10 may be receiving botulinum toxin injections intermittently at regular intervals (e.g., every three months) and, after reviewing the subject's 10 physical therapy information, the medical practitioner 30 may decide to make adjustments to the botulinum toxin treatment, such as adjusting the mode of administration, the frequency, or the amount of each dose, or selecting which muscle(s) to administer the botulinum toxin.

The medical practitioner 30 can access and review the subject's 10 physical therapy information during the subject's 10 clinic visit or in preparation for the subject's 10 next clinic visit. Having reviewed the physical therapy information and making a decision to adjust the botulinum toxin treatment, the medical practitioner 30 (or another medical professional) may administer the botulinum toxin to the subject 10 according to the adjustment in mode, dosage amount, frequency, muscle selection, etc.

It is to be understood that the present invention is not limited to the embodiments, aspects, or features described above, but encompasses any and all embodiments, aspects, and features within the scope of the following claims.

All references set forth above are incorporated herein by reference in their entireties.

1-77. (canceled)

78. A method for improving active movement capacity in a subject, the method comprising:
a) administering a first amount of botulinum toxin to the subject; and
b) administering a second amount of botulinum toxin to the subject in accordance with a treatment regimen, wherein the mode of administration, the dosage amount, the frequency of administration, and/or the muscles to which the botulinum toxin is administered is based, at least in part, on the type, duration, and/or frequency of physical activity undergone by the subject following administration of the first amount of botulinum toxin.

79-80. (canceled)

81. The method of claim 78, herein the first amount of botulinum toxin is administered to the subject within three months of the subject suffering a stroke.

82. The method of claim 78, wherein, before the subject exhibits symptoms of impaired active movement capacity, the subject receives an initial botulinum toxin treatment.

83. The method of claim 82, wherein the subject receives the initial botulinum toxin treatment within three months of suffering a stroke.

84. The method of claim 78, further comprising entering information related to the type, duration, and/or frequency of physical activity undergone by the subject following administration of the first amount of botulinum toxin into a software program configured to receive the information.

85. The method of claim 84, wherein the software program operates on a computer device.

86. The method of claim 84, wherein the information related to the type, duration, and/or frequency of physical activity undergone by the subject following administration of the first amount of botulinum toxin is stored in a database.

87-100. (canceled)

101. The method of claim 78, wherein the subject performs the physical activity on a daily basis.

102. The method of claim 78, wherein information related to the type, duration, and/or frequency of physical activity undergone by the subject following administration of the first amount of botulinum toxin is recorded on a daily basis.

103. A computer system programmed to perform steps of a computer-implemented method, the method comprising:
receiving information associated with a subject's physical therapy regimen from a subject who is undergoing both physical therapy and botulinum toxin treatment for impaired active movement capacity;
storing the physical therapy information in a database; and
transmitting the physical therapy information to a medical practitioner who utilizes the information in helping determine the mode of administration, the dosage amount, the frequency of administration, and/or the muscles to which to administer the botulinum toxin to the subject.

104. The computer system of claim 103, wherein the physical therapy information is received by the computer system from a first computer device, the database resides on a second computer device that is part of the computer system, and the physical therapy information is transmitted by the computer system to a third computer device.

108. A non-transitory computer-readable storage medium storing instructions that, when executed by a computer system, causes the computer system to perform steps of a computer-implemented method, the method comprising:
receiving, from a subject who is undergoing both physical therapy and botulinum toxin treatment for impaired active movement capacity, information associated with the physical therapy;
storing the physical therapy information in a database; and
transmitting the physical therapy information to a medical practitioner who utilizes the information in helping determine the mode of administration, the dosage amount, the frequency of administration, and/or the muscles to which to administer the botulinum toxin to the subject.

109. The method of claim 78, wherein one or both of the first and second amounts of botulinum toxin are injected into an affected muscle.

110. The method of claim 78, wherein one or both of the first and second amounts of botulinum toxin are administered into a neuromuscular junction.

111. The method of claim 78, wherein the physical therapy information comprises information related to the subject's symptoms associated with the impaired active movement capacity.

112. The method of claim 78, wherein the physical activity comprises: a) stretching the affected muscle; b) exercising the affected muscle; c) training the affected muscle; d) performing alternating movements of the affected muscle; e) heating or cooling the affected muscle; f) contracting or relaxing the affected muscle; and/or g) body positioning.

113. The method of claim 78, wherein information related to the type, duration, and/or frequency of physical activity undergone by the subject following administration of the first amount of botulinum toxin is received on a first computer device and is accessed on a second computer device.

114. The method of claim 78, wherein the second amount of botulinum toxin is determined based on an assessment of the subject's response following the first botulinum toxin treatment and the physical therapy.

115. The method of claim 84, further comprising administering to the subject a third amount of botulinum toxin, wherein the mode of administration, the dosage amount, the frequency of administration, and/or the muscles to which de third amount is administered is based, at least in part, on the information received by the software program.

116. The method of claim 78, wherein the subject has been instructed to: 1) undergo physical activity involving the affected muscle; and 2) record information related to the type, duration, and/or frequency of the subject's physical activity following administration of the first amount of botulinum toxin.
